(19) United States
(12) Patent Application Publication
(10) Pub. No.: US 2025/0137983 A1
(43) Pub. Date: May 1, 2025

(54) ATMOSPHERIC ENVIRONMENT ANALYSIS SYSTEM

(71) Applicant: Kioxia Corporation, Tokyo (JP)

(72) Inventors: Yui YASUDA, Kuwana Mie (JP); Hiroshi TOMITA, Yokkaichi Mie (JP); Naoki KANDA, Yokkaichi Mie (JP); Yoshio KASAI, Yokkaichi Mie (JP); Satoshi TAKAYAMA, Obu Aichi (JP)

(73) Assignee: Kioxia Corporation, Tokyo (JP)

(21) Appl. No.: 18/821,614

(22) Filed: Aug. 30, 2024

(30) Foreign Application Priority Data
Oct. 30, 2023 (JP) 2023-185639

(51) Int. Cl. G01N 33/00

(52) U.S. Cl. CPC G01N 33/0029; G01N 33/0014

(57) ABSTRACT

An analysis system includes an analysis device; a first pipe including a first end portion, a second end portion, and a first location between the first end portion and the second end portion; a first valve between the second end portion and the analysis device; a gas supply source supplying purge gas; a second pipe including a third end portion connected to the first location, and a fourth end portion connected to the gas supply source; a second valve in the second pipe; a third pipe including a fifth end portion, a sixth end portion, and a second location between the fifth end portion and the sixth end portion; a third valve between the sixth end portion and the analysis device; a fourth pipe including a seventh end portion connected to the second location, and an eighth end portion connected to the gas supply source; and a fourth valve.

### CROSS-REFERENCE TO RELATED APPLICATION(S)

This application is based upon and claims the benefit of priority from Japanese Patent Application No. 2023-185639, filed Oct. 30, 2023, the entire contents of which are incorporated herein by reference.

### FIELD

Embodiments described herein relate generally to an atmospheric environment analysis system.

### BACKGROUND

In a clean room used in a step of manufacturing a semiconductor device, a contaminant of ambient air may be floating. Therefore, in the clean room, the analysis of the ambient air is performed using an ion chromatography system or the like.

### DESCRIPTION OF THE DRAWINGS

FIG. 1 is a schematic diagram of an atmospheric environment analysis system according to a first embodiment.

FIG. 2 is a plan view conceptually showing a configuration example of a manufacturing system of semiconductor device disposed around the atmospheric environment analysis system of the first embodiment.

FIG. 3 is a plan view schematically showing a manufacturing apparatus 603 provided in the manufacturing system of semiconductor device of the first embodiment.

FIG. 4 is a perspective view schematically showing an EFEM unit provided in the manufacturing apparatus of the semiconductor device of the first embodiment.

FIG. 5 is a schematic diagram showing a dry etching unit as a first example of a main processing unit.

FIG. 6 is a schematic diagram showing a film forming unit (sputtering unit) as a second example of the main processing unit.

FIG. 7 is a flowchart of an atmospheric environment analysis method according to the first embodiment.

FIG. 8 is a flowchart of the atmospheric environment analysis method according to the first embodiment.

FIG. 9 is a diagram schematically showing a time change in a concentration at a first measurement location A1, a concentration at a second measurement location A2, an opening and closing of a first valve 52, an opening and closing of a second valve 54, an opening and closing of a third valve 56, and an opening and closing of a fourth valve 58 in the atmospheric environment analysis method according to the first embodiment.

FIG. 10 is a schematic diagram showing an example of a usage aspect of the atmospheric environment analysis system of the first embodiment.

FIG. 11 is a schematic diagram showing an example of a usage aspect of the atmospheric environment analysis system of the first embodiment.

FIG. 12 is a diagram schematically showing an elapse of time of analysis of a pipe and purge gas supply (cleaning) in the atmospheric environment analysis system according to the first embodiment.

FIG. 13 is a diagram schematically showing the elapse of time of analysis of the pipe and purge gas supply (cleaning) in the atmospheric environment analysis system of the first embodiment.

FIG. 14 is a diagram schematically showing the elapse of time of analysis of the pipe and purge gas supply (cleaning) in the atmospheric environment analysis system of the first embodiment.

FIG. 15 is a schematic diagram of an atmospheric environment analysis system of a second embodiment.

FIG. 16 is a flowchart of an atmospheric environment analysis method according to the second embodiment.

FIG. 17 is a flowchart of the atmospheric environment analysis method according to the second embodiment.

FIG. 18 is a schematic diagram showing an example of a usage aspect of the atmospheric environment analysis system of the second embodiment.

FIG. 19 is a schematic diagram showing an example of a usage aspect of the atmospheric environment analysis system of the second embodiment.

FIG. 20 is a schematic diagram of an atmospheric environment analysis system of a third embodiment.

FIG. 21 is a flowchart of an atmospheric environment analysis method according to a third embodiment.

FIG. 22 is a flowchart of the atmospheric environment analysis method according to the third embodiment.

FIG. 23 is a diagram schematically showing an elapse of time of analysis of a pipe and purge gas supply (cleaning) in the atmospheric environment analysis system according to a fourth embodiment.

FIG. 24 is a diagram schematically showing the elapse of time of analysis of the pipe and purge gas supply (cleaning) in the atmospheric environment analysis system according to the fourth embodiment.

FIG. 25 is a diagram schematically showing an elapse of time of analysis of a pipe and purge gas supply (cleaning) in the atmospheric environment analysis system according to a fifth embodiment.

FIG. 26 is a diagram schematically showing the elapse of time of analysis of the pipe and purge gas supply (cleaning) in the atmospheric environment analysis system according to the fifth embodiment.

### DETAILED DESCRIPTION

Embodiments provide an atmospheric environment analysis system that can efficiently perform environment analysis.

In general, according to one embodiment, an atmospheric environment analysis system includes an analysis device; a first pipe including a first end portion provided at a first measurement location, a second end portion, and a first location provided between the first end portion and the second end portion; a first valve provided between the second end portion of the first pipe and the analysis device; a gas supply source configured to supply purge gas; a second pipe including a third end portion connected to the first location of the first pipe, and a fourth end portion connected to the gas supply source; a second valve provided in the second pipe; a third pipe including a fifth end portion provided at a second measurement location, a sixth end portion, and a second location provided between the fifth end portion and the sixth end portion; a third valve provided between the sixth end portion of the third pipe and the analysis device; a fourth pipe including a seventh end portion connected to the second location of the third pipe, and an eighth end portion connected to the gas supply source; and a fourth valve provided in the fourth pipe.

Hereinafter, embodiments will be described with reference to drawings. In the drawings, the same or similar parts are denoted by the same or similar reference numerals.

### First Embodiment

According to one embodiment, an atmospheric environment analysis system includes an analysis device; a first pipe that includes a first end portion that is provided at a first measurement location, a second end portion, and a first location that is provided between the first end portion and the second end portion; a first valve that is provided between the second end portion of the first pipe and the analysis device; a gas supply source that supplies purge gas; a second pipe that includes a third end portion connected to the first location of the first pipe, and a fourth end portion connected to the gas supply source; a second valve that is provided in the second pipe; a third pipe that includes a fifth end portion that is provided at a second measurement location, a sixth end portion, and a second location provided between the fifth end portion and the sixth end portion; a third valve that is provided between the sixth end portion of the third pipe and the analysis device; a fourth pipe that includes a seventh end portion connected to the second location of the third pipe, and an eighth end portion connected to the gas supply source; and a fourth valve that is provided in the fourth pipe.

In addition, the atmospheric environment analysis system of the present embodiment further includes a control device that is capable of executing an atmospheric environment analysis method, in which the atmospheric environment analysis method includes supplying a first analysis target to the analysis device from the first measurement location via the first end portion, performing first analysis for the first analysis target by using the analysis device, after an elapse of a first predetermined time from a start of the first analysis, when a concentration of the first analysis target measured by the first analysis is equal to or higher than a first threshold concentration, closing the first valve, opening the second valve, and supplying the purge gas to the first measurement location from the gas supply source via the first end portion, supplying a second analysis target to the analysis device from the second measurement location via the fifth end portion, performing second analysis for the second analysis target by using the analysis device, and performing, when the concentration of the first analysis target measured by the first analysis is not equal to or higher than the first threshold concentration, the second analysis without supplying the purge gas.

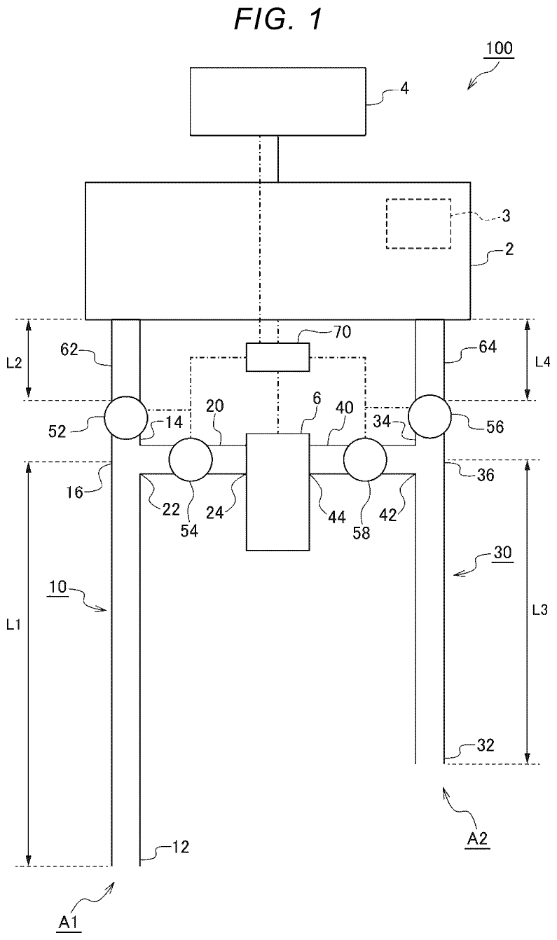

FIG. 1 is a schematic diagram of an atmospheric environment analysis system 100 of the present embodiment.

The atmospheric environment analysis system 100 is provided, for example, in a clean room. The atmospheric environment analysis system 100 is used, for example, for analyzing the ambient air in the clean room. The atmospheric environment analysis system 100 may be provided, for example, outside the clean room.

An analysis device 2 is, for example, an ion chromatograph. The analysis device 2 may be, for example, a device used for mass spectrometry, optical analysis such as ultraviolet absorption spectrophotometry or ultraviolet fluorescence spectrophotometry, or electrochemical analysis such as a solution conductivity method.

A first pipe 10 includes a first end portion 12 and a second end portion 14. The first end portion 12 is provided at, for example, a first measurement location A1 in a clean room. The first end portion 12 is open. In other words, the first end portion 12 is an open end of the pipe. The second end portion 14 is connected to the analysis device 2 by the first valve 52 and by the fifth pipe 62 that connects the first valve 52 to the analysis device 2.

A gas supply source 6 supplies purge gas. Here, the purge gas is, for example, a high-purity nitrogen gas or a clean dry air (CDA) gas. The gas supply source 6 may be, for example, a gas cylinder for storing nitrogen gas. The gas supply source 6 may be a gas supply source capable of supplying nitrogen gas generated by, for example, evaporating liquid nitrogen from a liquid nitrogen tank. As described above, the aspect of the gas supply source 6 is not particularly limited.

A second pipe 20 has a third end portion 22 and a fourth end portion 24. The third end portion 22 is connected to the first location 16 between the first end portion 12 and the second end portion 14 of the first pipe 10. The fourth end portion 24 is connected to the gas supply source 6.

It is preferable that a length L2 of the fifth pipe 62 is shorter than a length L1 of the first pipe 10 between the first end portion 12 and the first location 16. Cleaning the inside of the fifth pipe 62 with the purge gas is difficult. Therefore, when the length L2 of the fifth pipe 62 is too long, the amount of contaminants, which are adsorbed on the inner wall of the fifth pipe 62, in the ambient air increases, and there is a concern that an error may occur in the analysis result of the analysis device 2.

A second valve 54 is provided in the second pipe 20.

A third pipe 30 has a fifth end portion 32 and a sixth end portion 34. The fifth end portion 32 is provided at, for example, a second measurement location A2 in the clean room. The fifth end portion 32 is open. In other words, the fifth end portion 32 is an open end of the pipe. The sixth end portion 34 is connected to the analysis device 2 by the third valve 56 and by a sixth pipe 64 that connects the third valve 56 to the analysis device 2.

The fourth pipe 40 has a seventh end portion 42 and an eighth end portion 44. The seventh end portion 42 is connected to a second location 36 between the fifth end portion 32 and the sixth end portion 34 of the third pipe 30. The eighth end portion 44 is connected to the gas supply source 6.

It is preferable that a length L4 of the sixth pipe 64 is shorter than a length L3 of the third pipe 30 between the fifth end portion 32 and the second location 36. Cleaning the inside of the sixth pipe 64 with the purge gas is difficult. When the length L4 of the sixth pipe 64 is too long, the amount of contaminants, which are adsorbed on the inner wall of the sixth pipe 64, in the ambient air increases, and there is a concern that an error may occur in the analysis result of the analysis device 2.

The fourth valve 58 is provided in the fourth pipe 40.

A second vacuum pump (second pump) 3 is provided in the analysis device 2. The second vacuum pump 3 sucks and supplies the analysis target from the first measurement location A1 to the analysis device 2 via the first end portion 12. The supplied analysis target is analyzed by the analysis device 2. In addition, the second vacuum pump 3 sucks and supplies the analysis target from the second measurement location A2 to the analysis device 2 via the fifth end portion 32. The analysis target may be sucked and supplied into the analysis device 2 by using a first vacuum pump (first pump) 4 provided outside the analysis device 2. The first vacuum pump 4 is an example of a first pump. In addition, the second vacuum pump 3 is an example of a second pump.

A control device 70 is connected to, for example, the first valve 52, the second valve 54, the third valve 56, the fourth valve 58, the gas supply source 6, the analysis device 2, and the first vacuum pump 4. The control device 70 performs, for example, control of opening and closing of the first valve 52, the second valve 54, the third valve 56, and the fourth valve 58, control of the analysis device 2, control of the on/off of the first vacuum pump 4, and control of the on/off of the second vacuum pump 3.

The control device 70 is, for example, an electronic circuit. The control device 70 is, for example, a computer configured with a combination of hardware such as an arithmetic circuit and software such as a program.

The first pipe 10, the second pipe 20, the third pipe 30, the fourth pipe 40, the fifth pipe 62, and the sixth pipe 64 are, for example, pipes formed of a fluororesin. In addition, the first pipe 10, the second pipe 20, the third pipe 30, the fourth pipe 40, the fifth pipe 62, and the sixth pipe 64 are, for example, pipes in which the inside of a metal pipe is lined with a fluororesin. The pipes preferably used as the first pipe 10, the second pipe 20, the third pipe 30, the fourth pipe 40, the fifth pipe 62, and the sixth pipe 64 are not limited to the above.

The lengths of the fifth pipe 62 and the sixth pipe 64 are, for example, shorter than 1 m. It is preferable that the lengths of the fifth pipe 62 and the sixth pipe 64 are as short as possible.

The lengths of the second pipe 20 and the fourth pipe 40 are, for example, about several meters. However, for example, when the gas supply source 6 is a gas supply source capable of supplying nitrogen gas generated by evaporating liquid nitrogen from a liquid nitrogen tank, the length of the second pipe 20 and the fourth pipe 40 may be about several hundred meters.

The lengths of the first pipe 10 and the third pipe 30 are longer than the lengths of the second pipe 20, the fourth pipe 40, the fifth pipe 62, and the sixth pipe 64. In addition, for example, the length of the third pipe 30 is different from the length of the first pipe 10. For example, a length of the first pipe 10 is about 100 m, and a length of the third pipe 30 is about 50 m. The length of the third pipe 30 may be longer than the length of the first pipe 10.

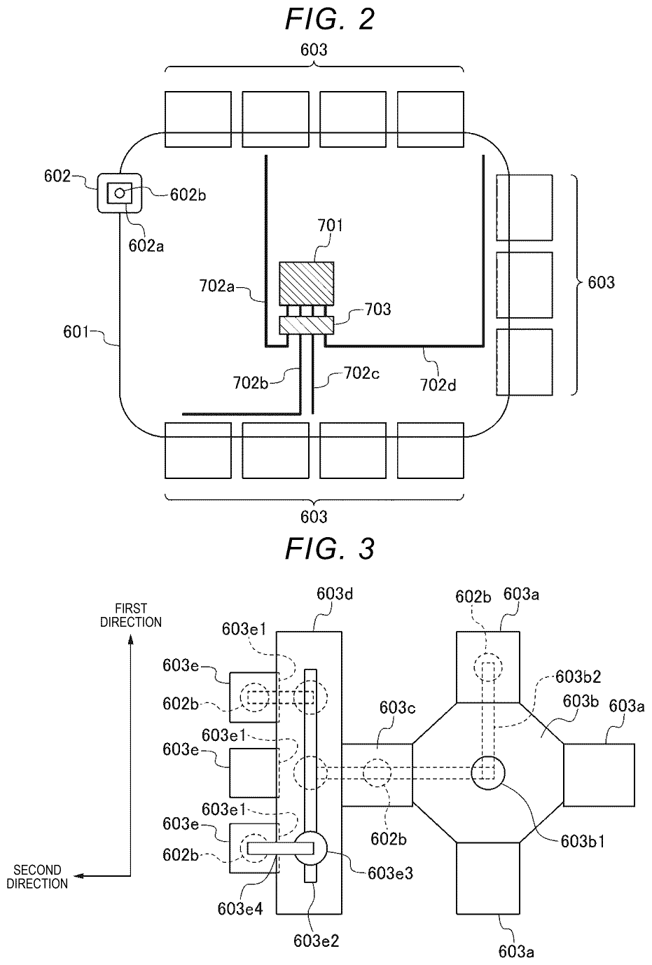

FIG. 2 is a plan view conceptually showing a configuration example of a manufacturing system of semiconductor device disposed around the atmospheric environment analysis system of the present embodiment.

A manufacturing system of semiconductor device of the present embodiment includes a track (ceiling track) 601, a conveyance vehicle (ceiling traveling conveyance vehicle) 602 that is movable along the track 601, a plurality of manufacturing apparatuses 603 disposed close to the track 601, and a measurement machine 701 for environmental evaluation.

The track 601 is provided on, for example, a ceiling of a manufacturing plant. In this case, the conveyance vehicle 602 functions as a ceiling traveling conveyance vehicle. The installation position of the track 601 is not limited to the ceiling. For example, the track 601 may be provided on the floor (ground) of the manufacturing plant or may be provided on a wall surface of the manufacturing plant. In addition, the conveyance vehicle 602 may not have wheels. In this case, the conveyance vehicle 602 may be driven, for example, in a linear motor type.

The conveyance vehicle 602 can be mounted with a front opening unified pod (FOUP) 602a. The FOUP 602a is mounted on the conveyance vehicle 602 in a state of storing a semiconductor wafer 602b, for example, and is conveyed.

The plurality of manufacturing apparatuses 603 are arranged along the track 601. Each manufacturing apparatus 603 includes a main processing unit 603a, a vacuum conveyance robot chamber 603b, a load lock chamber 603c, an EFEM unit 603d, and a load port unit 603e, as will be described later.

The measurement machine 701 is disposed, for example, in the vicinity of the center of the manufacturing system. A plurality of pipes 702a, 702b, 702c, and 702d are connected to the measurement machine 701. Each of tip portions (end portions) of the plurality of pipes 702a, 702b, 702c, and 702d are disposed at different locations in the manufacturing system. The measurement machine 701 is connected to base end portions of the plurality of pipes 702a, 702b, 702c, and 702d by a pipe cleaning mechanism 703.

The measurement machine 701 corresponds to the analysis device 2 of the present embodiment. In addition, the pipe 702a, the pipe 702b, the pipe 702c, and the pipe 702d correspond to the first pipe 10 or the third pipe 30 of the present embodiment. The pipe cleaning mechanism 703 corresponds to the gas supply source 6, the first valve 52, the second valve 54, the third valve 56, the fourth valve 58, the second pipe 20, the fourth pipe 40, the fifth pipe 62, the sixth pipe 64, and the control device 70 of the present embodiment.

FIG. 3 is a plan view schematically showing the manufacturing apparatus 603 provided in the manufacturing system of semiconductor device of the present embodiment.

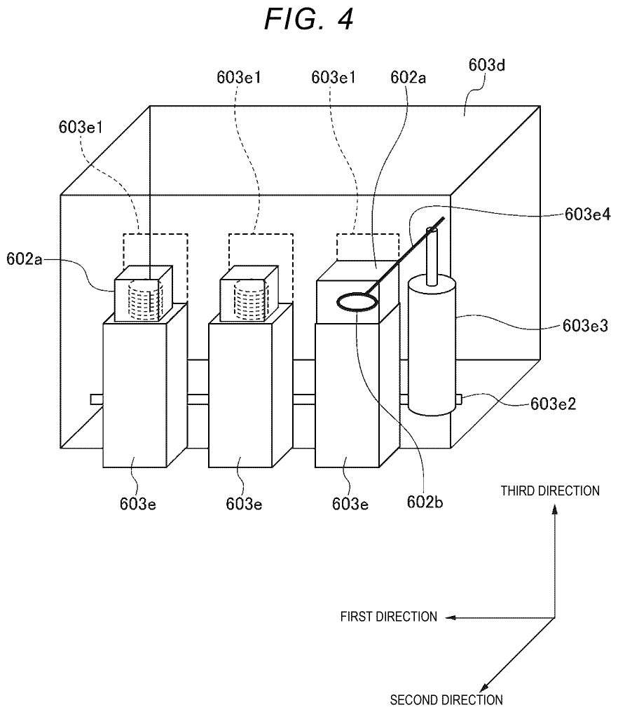

FIG. 4 is a perspective view schematically showing the EFEM unit 603d provided in the manufacturing apparatus 603 of the semiconductor device of the present embodiment.

Each manufacturing apparatus 603 includes the main processing unit 603a, the vacuum conveyance robot chamber 603b, the load lock chamber 603c, the EFEM unit 603d, and the load port unit 603e.

The main processing unit 603a is, for example, a dry etching unit or a film forming unit (a sputtering unit or a CVD unit), but is not limited thereto. The main processing unit 603a is connected to the vacuum conveyance robot chamber 603b.

A conveyance robot 603b1 is provided in the vacuum conveyance robot chamber 603b. A conveyance arm 603b2 is provided in the conveyance robot 603b1. The wafer 602b is taken in and out of the main processing unit 603a via the vacuum conveyance robot chamber 603b.

The wafer 602b is transferred between the vacuum conveyance robot chamber 603b and the EFEM unit 603d via the load lock chamber 603c.

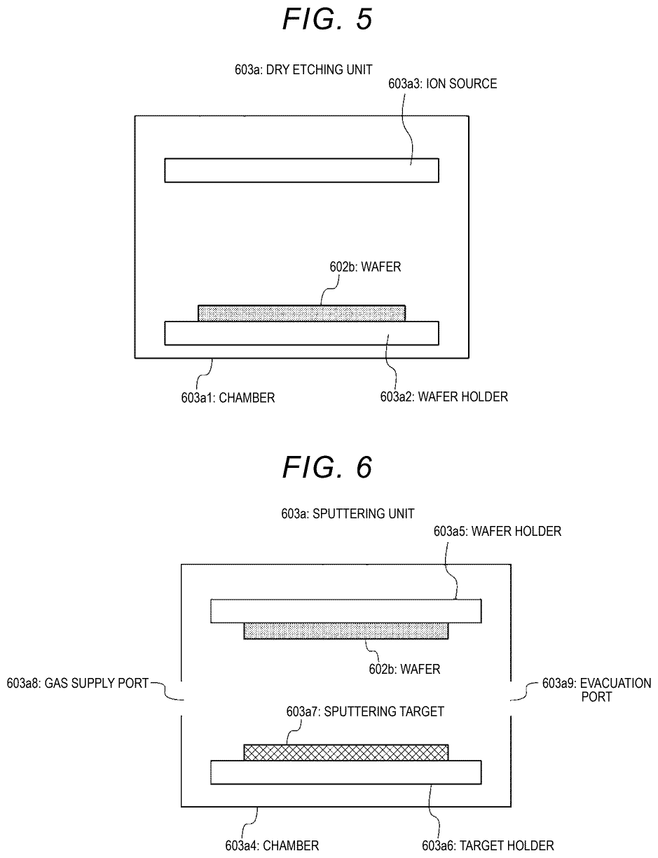

FIG. 5 is a schematic diagram showing a dry etching unit as a first example of the main processing unit 603a. The dry etching unit includes, for example, a chamber 603al, a wafer holder 602a2, and an ion source 603a3. A wafer holder 603a2 holds the wafer 602b accommodated in the chamber 603al. The ion source 603a3 performs dry etching of the wafer 602b by irradiating the wafer 602b with ions.

FIG. 6 is a diagram showing a film forming unit (sputtering unit) as a second example of the main processing unit 603a. The film forming unit (sputtering unit) includes a chamber 603a4, a wafer holder 603a5, and a target holder 603a6. The wafer holder 603a5 holds the wafer 602b. The target holder 603a6 holds a sputtering target 603a7. The chamber 603a4 is provided with a gas supply port 603a8 for supplying a film-forming gas and an evacuation port 603a9 for discharging unnecessary gas.

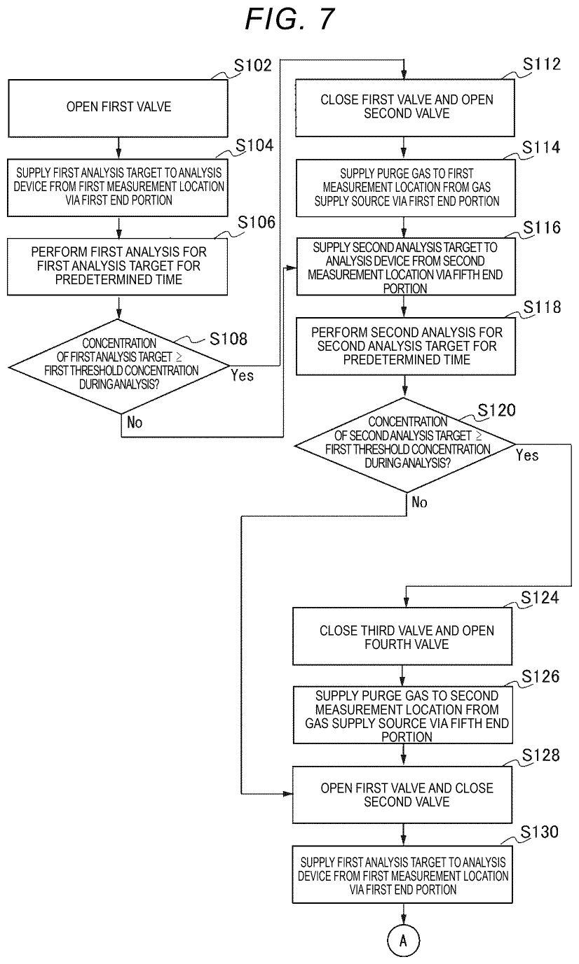

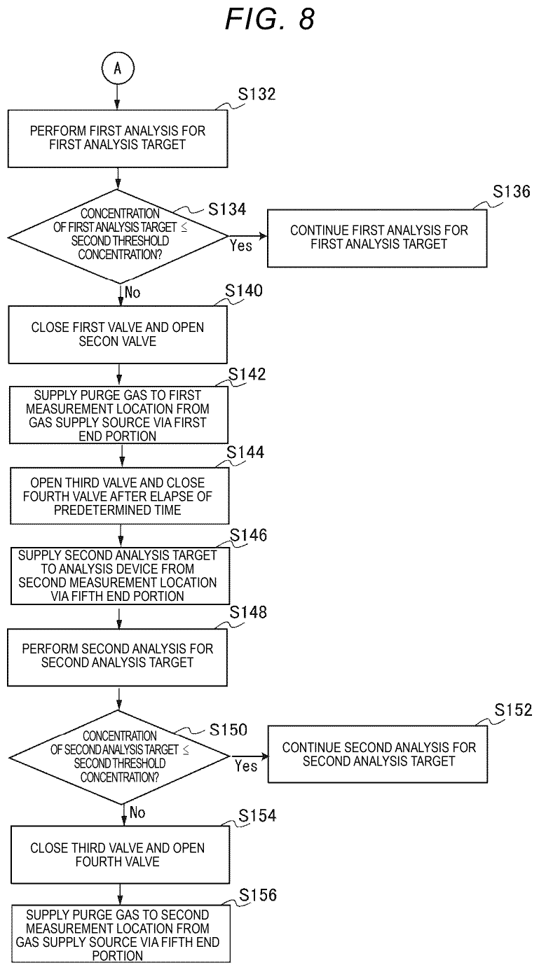

FIGS. 7 and 8 are flowcharts of the atmospheric environment analysis method of the present embodiment.

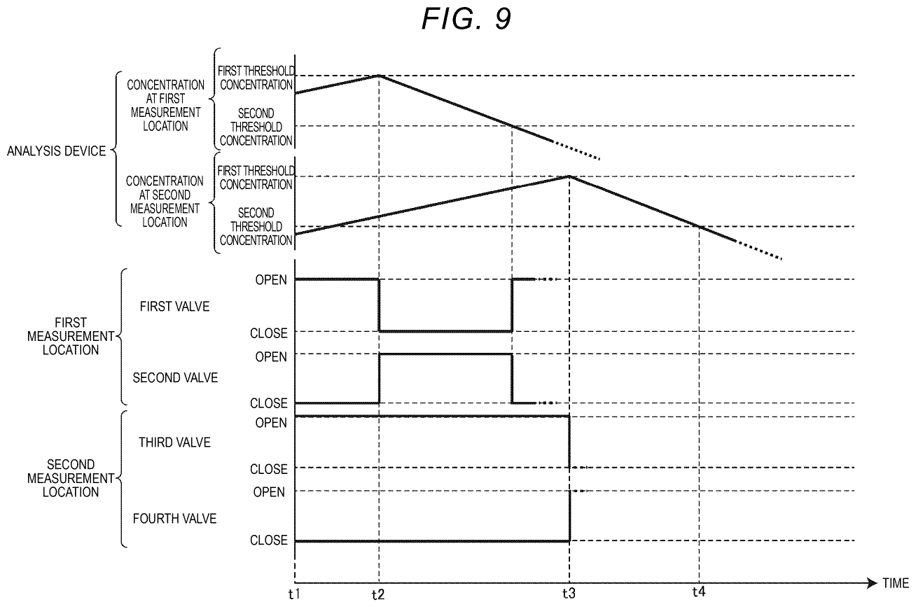

FIG. 9 is a diagram schematically showing a time change in a concentration at the first measurement location A1, a concentration at the second measurement location A2, an opening and closing of the first valve 52, an opening and closing of the second valve 54, an opening and closing of the third valve 56, and an opening and closing of the fourth valve 58 in the atmospheric environment analysis method according to the present embodiment.

First, the first valve 52 is opened (S102 in FIG. 7, time t1 in FIG. 9). It is assumed that the second valve 54 is closed. In addition, it is assumed that the third valve 56 is open. The third valve 56 may be closed. In addition, it is assumed that the fourth valve 58 is closed. The fourth valve 58 may be open.

Next, the analysis target is supplied to the analysis device 2 from the first measurement location A1 via the first end portion 12 by using the first vacuum pump 4 or the second vacuum pump 3 (S104 in FIG. 7). Here, the analysis target at the first measurement location A1 is referred to as a first analysis target.

Next, the analysis device 2 is used to analyze the first analysis target for a predetermined time (an example of the first predetermined time) (first analysis) (S106 in FIG. 7).

For example, when the concentration of the first analysis target at the first measurement location A1 is high, it is considered that the concentration of the analysis target measured by the first analysis increases as time elapses.

Then, during the analysis (first analysis), when the concentration of the first analysis target measured by the first analysis is equal to or higher than the first threshold concentration, the first valve 52 is closed and the second valve 54 is opened (S108 and S112 in FIG. 7 and time t2 in FIG. 9). Next, the purge gas is supplied to the first measurement location A1 from the gas supply source 6 via the second valve 54 and the first end portion 12 (S114 in FIG. 7). Next, the analysis target is supplied to the analysis device 2 from the second measurement location A2 via the fifth end portion 32 (S116 in FIG. 7). Here, the analysis target at the second measurement location A2 is referred to as a second analysis target. When the third valve 56 is closed, the third valve 56 is opened. Further, when the fourth valve 58 is open, the fourth valve 58 is closed. In addition, when the concentration of the first analysis target measured by the first analysis is lower than the first threshold concentration, the second analysis target is supplied to the analysis device 2 from the second measurement location A2 via the fifth end portion 32 (S116).

Next, the analysis device 2 is used to analyze the second analysis target (second analysis) for a predetermined time (an example of a second predetermined time, and the first predetermined time and the second predetermined time may be the same or different) (S118 in FIG. 7).

For example, when the concentration of the second analysis target at the second measurement location A2 is high, it is considered that the concentration of the analysis target measured by the second analysis increases as time elapses.

Then, during the analysis (second analysis), when the concentration of the second analysis target measured by the second analysis is equal to or higher than the first threshold concentration, the third valve 56 is closed, the fourth valve 58 is opened (S120 and S124 in FIG. 7 and time t3 in FIG. 9), and the purge gas is supplied to the second measurement location A2 from the gas supply source 6 via the fifth end portion 32 (S126 in FIG. 7). Further, when the first valve 52 is not open, the first valve 52 is opened, and when the second valve 54 is not closed, the second valve 54 is closed, and the first analysis target is supplied to the analysis device 2 from the first measurement location A1 via the first end portion 12 (S128 and S130 in FIG. 7). Then, the first analysis of the first analysis target is performed (S132 in FIG. 8). When the concentration of the second analysis target measured by the second analysis is not equal to or higher than the first threshold concentration, the first valve 52 is opened if the first valve 52 is not opened, and the second valve 54 is closed if the second valve 54 is not closed (S128 in FIG. 7).

Next, when the concentration of the first analysis target is equal to or lower than the second threshold concentration which is lower than the first threshold concentration, the first analysis is continued (S134 and S136 in FIG. 8). When the concentration of the first analysis target is not equal to or lower than the second threshold concentration, the first valve 52 is closed, the second valve 54 is opened, and the purge gas is supplied to the first measurement location A1 from the gas supply source 6 via the first end portion 12 (S140 and S142 in FIG. 8).

Next, after an elapse of a predetermined time (an example of a third predetermined time, and the first predetermined time, the second predetermined time, and the third predetermined time may be equal to or different from each other), when the third valve 56 is not open, the third valve 56 is opened, and when the fourth valve 58 is not closed, the fourth valve 58 is closed, and the second analysis target is supplied to the analysis device 2 from the second measurement location A2 via the fifth end portion 32. Then, the second analysis for the second analysis target is performed (S144, S146, and S148 in FIG. 8).

When the concentration of the second analysis target measured by the second analysis is equal to or lower than the second threshold concentration, the second analysis is continued (time t4 in FIG. 9, and S150 and S152 in FIG. 8). When the concentration of the second analysis target measured by the second analysis is not equal to or lower than the second threshold concentration, the third valve 56 is closed, the fourth valve 58 is opened, and the purge gas is supplied to the second measurement location A2 from the gas supply source 6 via the fourth valve 58 and the fifth end portion 32 (S154 and S156 in FIG. 8).

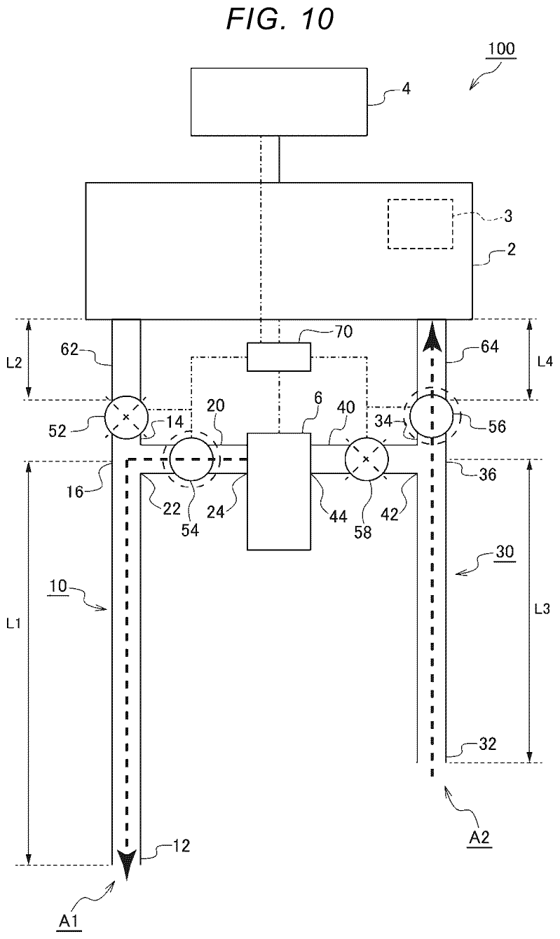

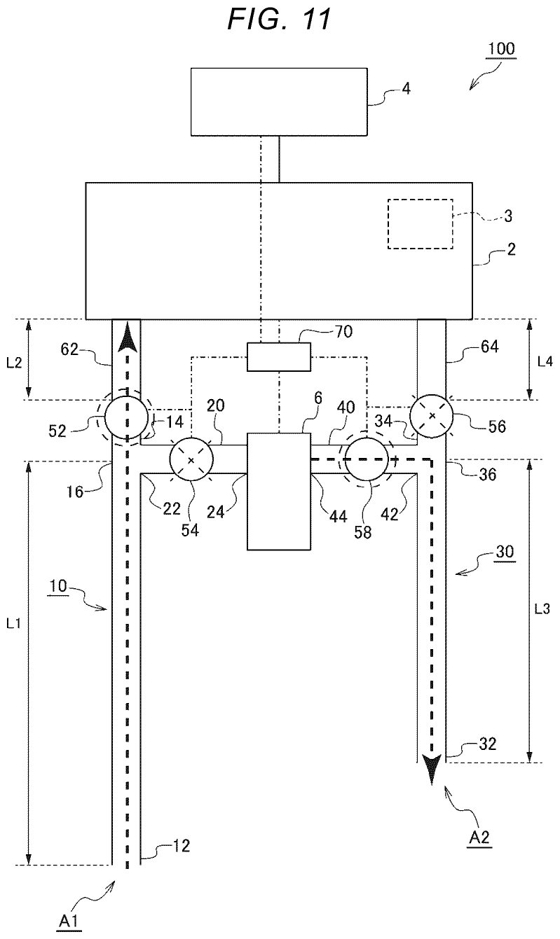

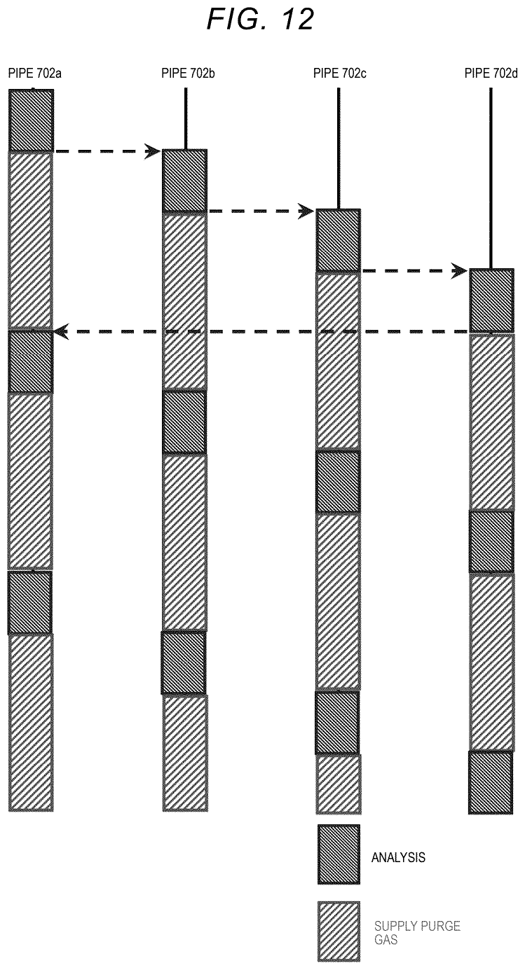

FIG. 10 is a schematic diagram showing an example of a usage aspect of the atmospheric environment analysis system of the present embodiment.

As shown in FIG. 10, the steps shown in FIG. 7, that is, the steps S114, S116, and S118 may be performed simultaneously in order to perform the environment analysis in a short time. Here, the cross-marks x in the dashed lines attached to the first valve 52 and the fourth valve 58 in FIG. 10 indicate that the first valve 52 and the fourth valve 58 are closed. In addition, the circles (o) in the dashed lines attached to the second valve 54 and the third valve 56 in FIG. 10 indicate that the second valve 54 and the third valve 56 are open. In addition, the arrows in the dashed lines in FIG. 10 indicate the flow of the air flow, the arrows being provided on the first pipe 10, the second pipe 20, and the third pipe 30 between the first end portion 12 and the first location 16. Similarly, the steps shown in FIGS. 8, S142, S146, and

S148 may be performed simultaneously in order to perform the environment analysis in a short time.

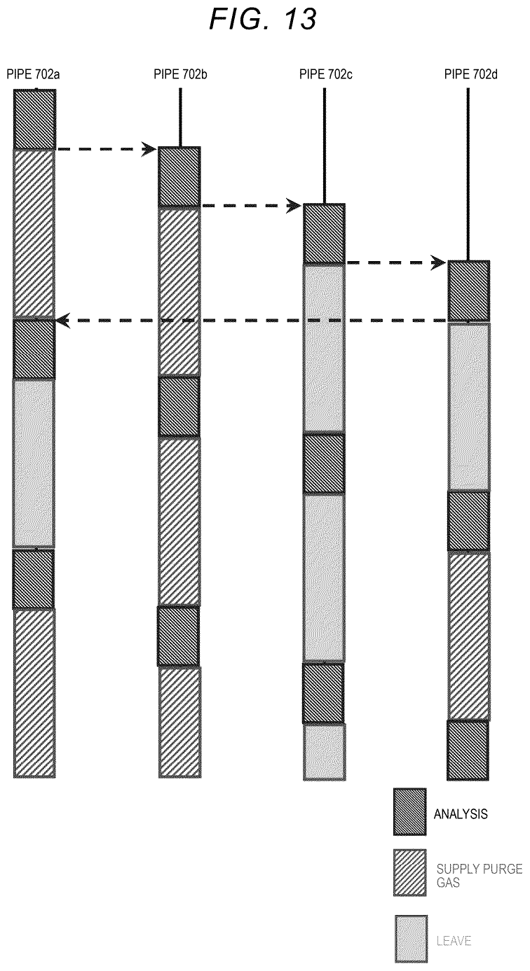

FIG. 11 is a schematic diagram showing an example of a usage aspect of the atmospheric environment analysis system of the present embodiment.

As shown in FIG. 11, the steps shown in S126 and S130 in FIG. 7 and the step shown in S132 in FIG. 8 may be performed simultaneously in order to perform the environment analysis in a short time. Here, the circles (o) in a dashed line attached to the first valve 52 and the fourth valve 58 in FIG. 11 indicate that the first valve 52 and the fourth valve 58 are open. In addition, the cross-marks (x, cross-mark) in the dashed line attached to the second valve 54 and the third valve 56 indicate that the second valve 54 and the third valve 56 are closed. In addition, arrows in the dashed lines attached to the first pipe 10, the fourth pipe 40, and the third pipe 30 between the fifth end portion 32 and the second location 36 indicate the flow of the air flow.

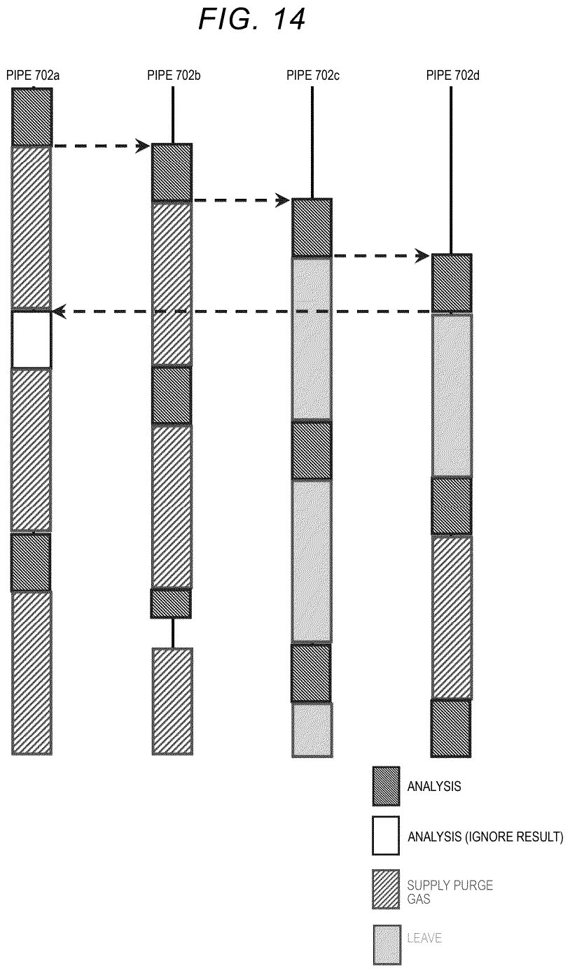

FIG. 12 is a diagram schematically showing an elapse of time of analysis of a pipe and purge gas supply (cleaning) in the atmospheric environment analysis system of the present embodiment. In FIG. 12, the time elapsing from the top to the bottom of the drawing is schematically shown.

The analysis is performed in order using the pipe 702a, the pipe 702b, the pipe 702c, and the pipe 702d in FIG. 2. Then, in each pipe, the purge gas is supplied while the analysis is not performed.

FIG. 13 is a diagram schematically showing an elapse of time of analysis of a pipe and purge gas supply (cleaning) in the atmospheric environment analysis system of the present embodiment. In FIG. 13, the time elapsing from the top to the bottom of the drawing is schematically shown.

When the analysis is completed, the purge gas is supplied when the concentration of the analysis target is equal to or higher than the first threshold concentration, and the purge gas supply is skipped when the concentration is lower than the first threshold concentration. By skipping the purge gas supply, the power consumption of the purge gas and the atmospheric environment analysis system can be reduced. When the purge gas supply is skipped, the opening and closing state of the valve is the same as when the analysis is performed. For example, in the system of the first pipe 10 in FIG. 1, leave the second valve 54 closed and the first valve 52 opened. In this way, the ambient air at the first measurement location flows in the analysis device 2 via the first pipe 10, so that there is a minimum cleaning effect.

FIG. 14 is a diagram schematically showing an elapse of time of analysis of a pipe and purge gas supply (cleaning) in the atmospheric environment analysis system of the present embodiment. In FIG. 14, the time elapsing from the top to the bottom of the drawing is schematically shown.

The concentration may be measured even during the purge gas supply. When the concentration of the analysis target does not fall below the first threshold concentration even when the purge gas is supplied, the analysis result of the next cycle may be ignored. In that case, it is expected that the concentration of the analysis target will be below the first threshold concentration in the subsequent purge gas supply, but when the concentration is not below the first threshold concentration, the purge gas supply is repeated in the same manner.

In the analysis, when the concentration of the analysis target is equal to or higher than the third threshold concentration which is higher than the first threshold concentration, the analysis may be ended early. When the concentration of the analysis target is significantly high, the analysis can be terminated early to prevent contamination of the analysis device and the pipe. In this case, the analysis using the next pipe is performed after a predetermined time elapsed.

Next, the effects of the atmospheric environment analysis device and the atmospheric environment analysis method of the present embodiment will be described.

The inside of the analysis device 2 such as the ion chromatography system is contaminated by the analysis target of the ambient air, which is the contaminant of the clean room. Therefore, it is conceivable that the inside of the analysis device 2 is cleaned with a purge gas such as nitrogen gas. Thereby, the contaminant inside the analysis device 2 can be removed.

However, since the analysis device 2 is connected to the measurement location by a pipe, the inner wall of the pipe connected to the analysis device 2 may also be adsorbed with the contaminant. When the contaminant is adsorbed on the inner wall of the pipe, there is a possibility that the measurement of the analysis target cannot be accurately performed in the analysis device 2. For example, after cleaning the inside of the analysis device 2, the contaminant may be removed by the gas flowing through the pipe by idling. However, in this case, it takes time to remove the contaminant adsorbed on the inner wall of the pipe. For example, when the analysis device is provided in a clean room for manufacturing a semiconductor device, the clean room is generally very large, and the length of the pipe may be, for example, about several hundred meters. Therefore, it takes more and more time to remove the contaminant adsorbed on the inner wall of the pipe.

In addition, it is assumed that a plurality of pipes are connected to the analysis device 2 in order to analyze the analysis target at a plurality of locations in the clean room. In this case, the distances between the analysis device 2 and the plurality of places where the analysis is performed are different from each other. Further, the lengths of the plurality of pipes are different from each other. As a result, the amount of the contaminants adsorbed on the inner wall may be different for each pipe, and it may not be possible to appropriately compare the analysis results of the analysis target at a plurality of locations.

Therefore, the atmospheric environment analysis system of the present embodiment has a mechanism for cleaning by supplying a purge gas to a pipe connecting the analysis device and a location where the analysis is performed.

By providing a valve (first valve and third valve) that may be disconnected from a gas supply source for each of the plurality of pipes (first pipe and third pipe), for example, the first pipe may be cleaned while the analysis of the second measurement location is performed using the third pipe. Meanwhile, for example, the third pipe may be cleaned while the analysis of the first measurement location is performed using the first pipe.

In addition, since the cleaning is performed with the purge gas when the concentration of the analysis target measured by the analysis is equal to or higher than the first threshold concentration, the cleaning may be performed without interfering with the efficiency of the measurement.

In addition, since the inner wall of the pipe may be cleaned, even if the pipes have different lengths, the adsorption states of the contaminants may be aligned (the contaminants adsorbed on the inner wall of the pipe may be removed to an extent that does not affect the measurement by the analysis device), and thus, the measurement results may be compared with each other.

According to the atmospheric environment analysis system and the atmospheric environment analysis method of the present embodiment, an atmospheric environment analysis system and an atmospheric environment analysis method capable of efficiently performing environment analysis can be provided.

### Second Embodiment

According to one embodiment, an atmospheric environment analysis system includes an analysis device, a first pipe that is provided at a first measurement location and includes a first end portion which is an open end and a second end portion which is connected to the analysis device; a first valve that is provided in the first pipe; a second pipe that includes a third end portion connected to a first location of the first pipe between the first valve and the second end portion, and includes an open fourth end portion; a second valve that is provided in the second pipe; a first filter that is provided in the second pipe between the second valve and the fourth end portion; a third pipe that is provided at a second measurement location and includes a fifth end portion which is an open end and a sixth end portion which is connected to the analysis device; a third valve that is provided in the third pipe; a fourth pipe that includes a seventh end portion which is connected to a second location of the third pipe between the third valve and the sixth end portion and an open eighth end portion; a fourth valve that is provided in the fourth pipe; a second filter that is provided in the fourth pipe between the fourth valve and the eighth end portion; and a first pump that is connected to the second end portion and the sixth end portion.

Here, description of content that overlaps with the first embodiment will be omitted.

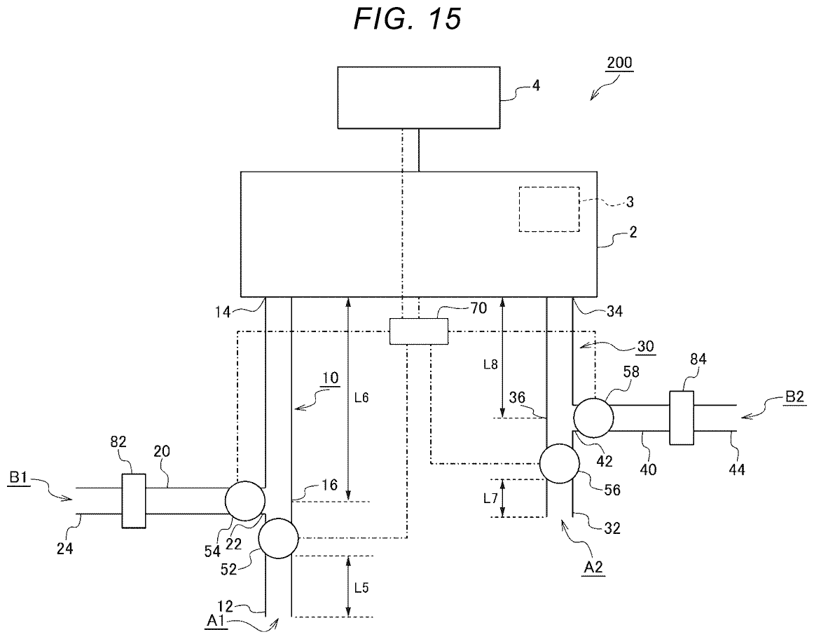

FIG. 15 is a schematic diagram of an atmospheric environment analysis system 200 of the present embodiment.

The second end portion 14 of the first pipe 10 is connected to the analysis device 2.

The second pipe 20 includes the third end portion 22 connected to the first location 16 of the first pipe 10 between the first valve 52 and the second end portion 14, and the open fourth end portion 24. The fourth end portion 24 is provided in, for example, the first environment B1 in the clean room. A first ambient air is supplied from the first environment B1 to the second end portion 14 via the fourth end portion 24. It is preferable that the first ambient air is taken in by the first vacuum pump 4. Generally, the evacuation capacity (capacity) of the second vacuum pump 3 provided inside the analysis device 2 is small, and thus, the first ambient air is supplied by using the first vacuum pump 4 having a larger evacuation capacity, so that the cleaning of the pipe may be completed in a shorter time. The intake of the first ambient air may be performed by the second vacuum pump 3.

A first filter 82 is provided between the second valve 54 and the fourth end portion 24. The first ambient air is filtered by the first filter 82.

The sixth end portion 34 of the third pipe 30 is connected to the analysis device 2.

The fourth pipe 40 includes the seventh end portion 42 connected to the second location 36 of the third pipe 30 between the third valve 56 and the sixth end portion 34, and the open eighth end portion 44. The eighth end portion 44 is provided in, for example, a second environment B2 in the clean room. The second ambient air is taken into the sixth end portion 34 from the second environment B2 via the eighth end portion 44. It is preferable that the second ambient air is taken in by the first vacuum pump 4. The second ambient air may be taken in by the second vacuum pump 3.

A second filter 84 is provided between the fourth valve 58 and the eighth end portion 44. The second ambient air is filtered by the second filter 84.

The first filter 82 and the second filter 84 are, for example, filters that can be mounted on a pipe and have a membrane made of an ion exchange resin, activated carbon, polytetrafluoroethylene (PTFE), or the like. The first filter 82 and the second filter 84 function as a filter unit. In other words, the atmospheric environment analysis system 200 of the present embodiment includes the first filter 82 and the second filter 84 as the filter unit.

In the atmospheric environment analysis system and the atmospheric environment analysis method of the present embodiment, the inner wall of the pipe is cleaned using the ambient air purified by the filter.

A length L6 of the first pipe 10 between the second end portion 14 and the first location 16 is preferably longer than a length L5 of the first pipe 10 between the first end portion 12 and the first valve 52. Cleaning of the first pipe 10 between the first end portion 12 and the first valve 52 by the first ambient air is difficult. Therefore, when the length L5 of the first pipe 10 between the first end portion 12 and the first valve 52 is too long, the amount of contaminants, which is adsorbed on the inner wall of the first pipe 10 between the first end portion 12 and the first valve 52, in the ambient air increases, and there is a concern that an error may occur in the analysis result of the analysis device 2.

A length L8 of the third pipe 30 between the sixth end portion 34 and the second location 36 is preferably longer than a length L7 of the third pipe 30 between the fifth end portion 32 and the third valve 56. Cleaning of the third pipe 30 between the fifth end portion 32 and the third valve 56 by the second ambient air is difficult. Therefore, when the length L7 of the third pipe 30 between the fifth end portion 32 and the third valve 56 is too long, the amount of contaminants, which is adsorbed on the inner wall of the third pipe 30 between the fifth end portion 32 and the third valve 56, in the ambient air increases, and there is a concern that an error may occur in the analysis result of the analysis device 2.

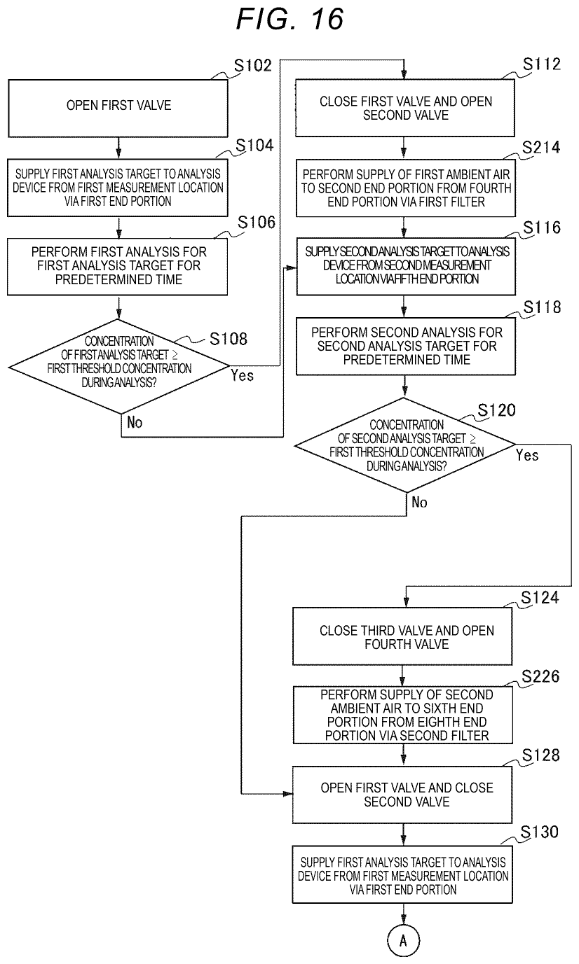

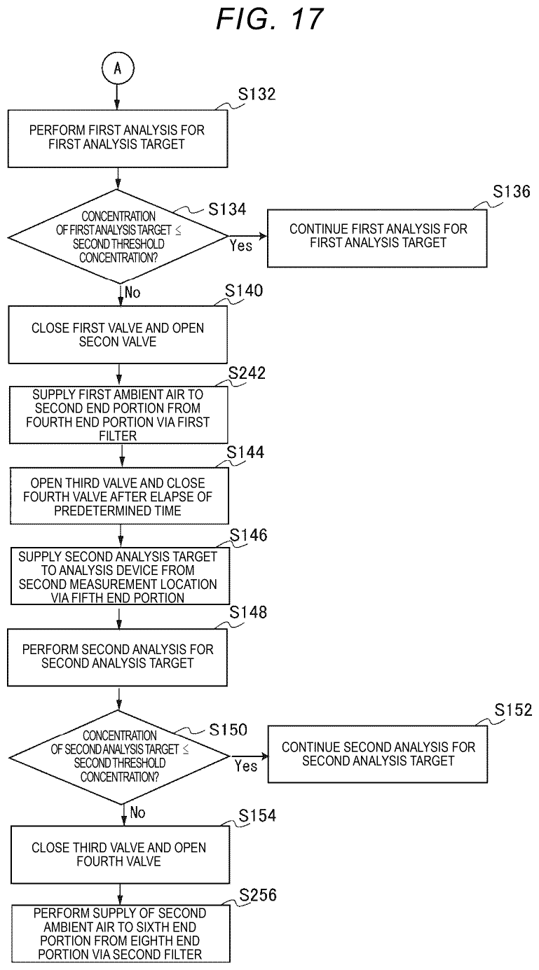

FIGS. 16 and 17 are flowcharts of the atmospheric environment analysis method of the present embodiment.

The difference from the flowchart of the first embodiment is that the purge gas supply to the first measurement location A1 from the gas supply source 6 via the second valve 54 and the first end portion 12 (S114 of FIG. 7 and S142 of FIG. 8) is replaced by the supply of a first ambient air to the second end portion 14 from the fourth end portion 24 via the first filter 82 and the third end portion 22 (S214 of FIGS. 16 and S242 of FIG. 17), and the purge gas supply to the second measurement location A2 from the gas supply source 6 via the fifth end portion 32 (S126 of FIGS. 7 and S156 of FIG. 8) is replaced by the supply of the second ambient air to the sixth end portion 34 from the eighth end portion 44 via the second filter 84 and the seventh end portion 42 (S226 of FIGS. 16 and S256 of FIG. 17).

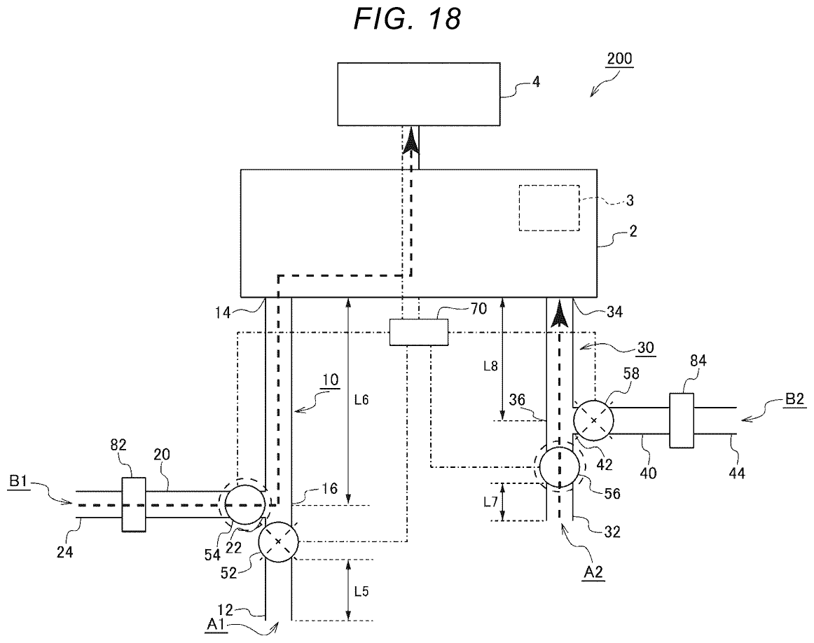

FIG. 18 is a schematic diagram showing an example of a usage aspect of the atmospheric environment analysis system of the present embodiment.

As shown in FIG. 18, the steps S214, S116, and S118 shown in FIG. 16 may be performed simultaneously in order to perform the environment analysis in a short time. Here, the cross-marks (x, cross-mark) in the dashed line attached to the first valve 52 and the fourth valve 58 in FIG. 18 indicate that the first valve 52 and the fourth valve 58 are closed. In addition, the circles (o) in the dashed line attached to the second valve 54 and the third valve 56 in FIG. 18 indicate that the second valve 54 and the third valve 56 are open. In addition, arrows in the dashed lines attached to the second pipe 20, the first pipe 10 between the first location 16 and the second end portion 14, the analysis device 2, between the first vacuum pump 4 and the analysis device 2, and the third pipe 30 in FIG. 18 indicate the flow of the air flow.

Similarly, the steps S242, S146, and S148 shown in FIG. 17 may be performed simultaneously in order to perform the environment analysis in a short time.

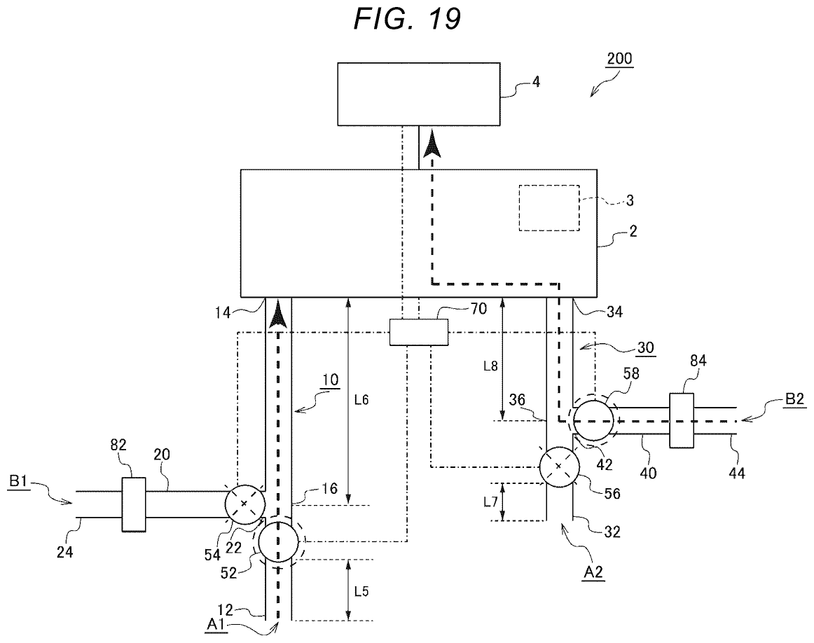

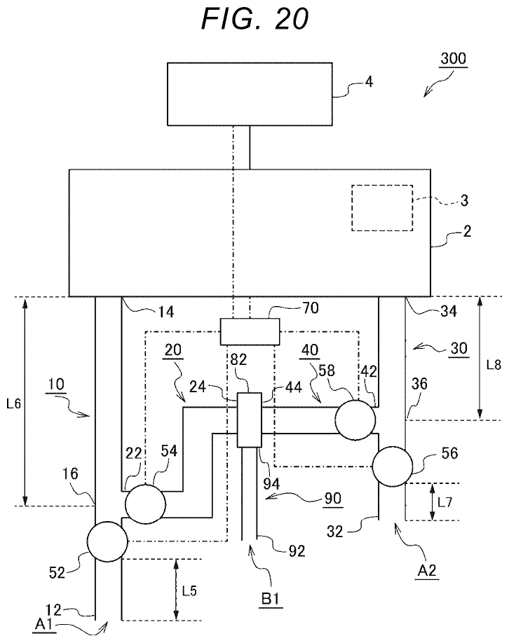

FIG. 19 is a schematic diagram showing an example of a usage aspect of the atmospheric environment analysis system of the present embodiment.

As shown in FIG. 19, the steps S226 and S130 shown in FIG. 16 and the step S132 shown in FIG. 17 may be performed simultaneously in order to perform the environment analysis in a short time. Here, the circles (o) in the dashed line attached to the first valve 52 and the fourth valve 58 in FIG. 19 indicate that the first valve 52 and the fourth valve 58 are open. In addition, the cross-marks (x, cross-mark) in the dashed line attached to the second valve 54 and the third valve 56 indicate that the second valve 54 and the third valve 56 are closed. In addition, arrows in the dashed lines attached to the first pipe 10, the fourth pipe 40, the third pipe 30 between the second location 36 and the sixth end portion 34, the analysis device 2, and between the first vacuum pump 4 and the analysis device 2 in FIG. 19 indicate the flow of the air flow.

As in the present embodiment, the cleaning of the inner wall of the pipe may be performed using the ambient air that passed through the filter.

According to the atmospheric environment analysis system and the atmospheric environment analysis method of the present embodiment, an atmospheric environment analysis system and an atmospheric environment analysis method capable of efficiently performing environment analysis can be provided.

### Third Embodiment

According to one embodiment, an atmospheric environment analysis system includes an analysis device; a first pipe that is provided at a first measurement location and includes a first end portion which is an open end portion and a second end portion which is connected to the analysis device; a first valve that is provided in the first pipe; a third pipe that is provided at a second measurement location and includes a fifth end portion which is an open end portion and a sixth end portion which is connected to the analysis device; a third valve that is provided in the third pipe; a second pipe that includes a third end portion connected to a first location of the first pipe between the first valve and the second end portion, and a fourth end portion connected to a second location of the third pipe between the third valve and the sixth end portion; a second valve that is provided in the second pipe; a fourth valve that is provided between the second valve and the second location of the second pipe; a first filter that is provided in the second pipe between the second valve and the fourth valve; a seventh pipe that includes a ninth end portion which is an open end and a tenth end portion which is connected to the first filter; and a first pump that is connected to the second end portion and the sixth end portion.

Here, the description of the content that overlaps with the first embodiment and the second embodiment is omitted.

FIG. 20 is a schematic diagram of the atmospheric environment analysis system 300 according to the present embodiment.

The third end portion 22 of the second pipe 20 is connected to the first location 16 of the first pipe 10 between the first valve 52 and the second end portion 14, and the fourth end portion 24 of the second pipe 20 is connected to the first filter 82 as a filter unit. In addition, the seventh end portion 42 of the fourth pipe 40 is connected to the second location 36 of the third pipe 30 between the third valve 56 and the sixth end portion 34, and the eighth end portion 44 of the fourth pipe 40 is connected to the first filter 82 as a filter unit.

The fourth valve 58 is provided between the second valve 54 and the second location 36 of the second pipe 20.

The first filter 82 is provided in the second pipe 20 between the second valve 54 and the fourth valve 58.

A seventh pipe 90 has a ninth end portion 92 which is an open end and is disposed in the first environment B1, and a tenth end portion 94 which is connected to the first filter 82. The seventh pipe 90 is, for example, a pipe formed of a fluororesin. In addition, the seventh pipe 90 is, for example, a pipe in which the inside of a metal pipe is lined with a fluororesin. The pipe preferably used as the seventh pipe 90 is not limited to the above.

The atmospheric environment analysis system 300 of the present embodiment is different from the atmospheric environment analysis system 200 of the second embodiment, and performs cleaning of the inner wall of the pipe by supplying the first ambient air filtered by one first filter 82 to the second end portion 14 and the sixth end portion 34. In other words, while the filter unit of the atmospheric environment analysis system 200 of the second embodiment includes the first filter 82 and the second filter 84, the filter unit of the atmospheric environment analysis system 300 of the third embodiment includes only the first filter 82.

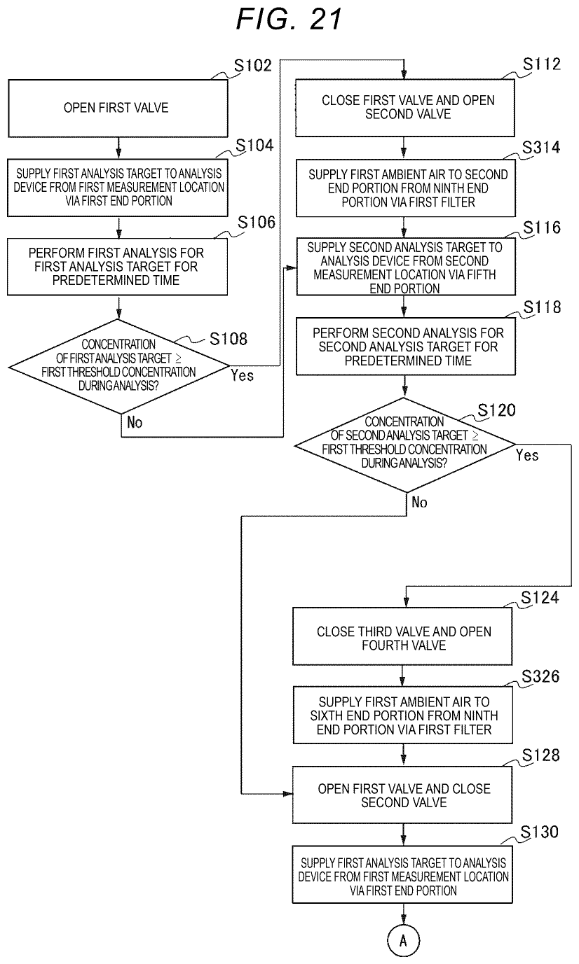

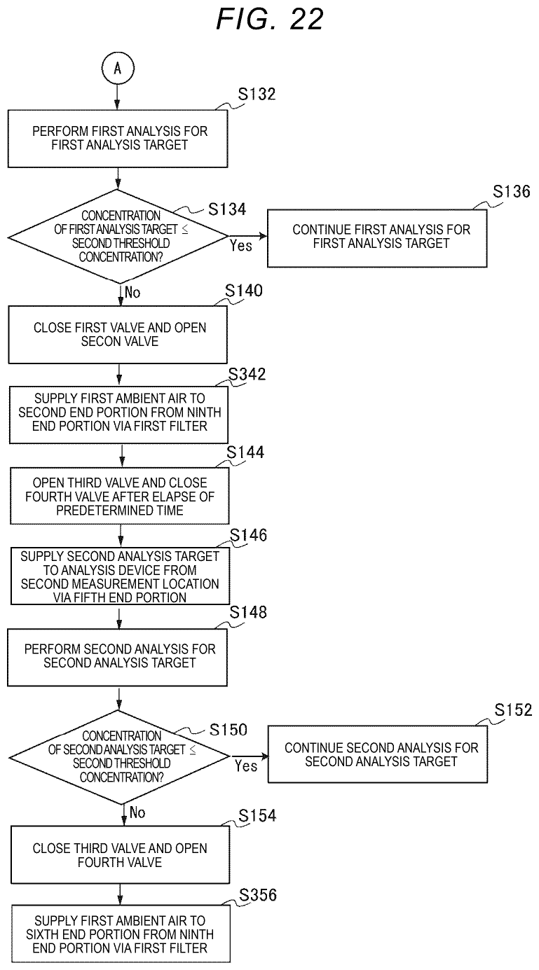

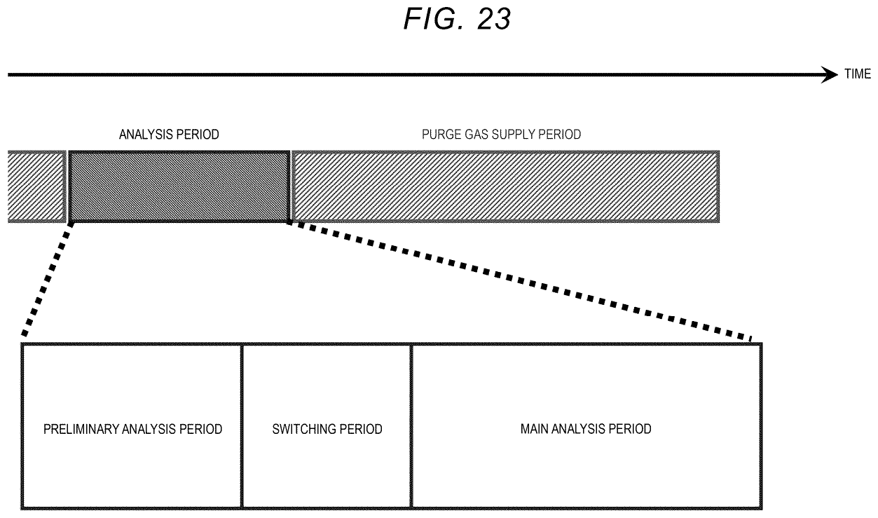

FIGS. 21 and 22 are flowcharts of the atmospheric environment analysis method of the present embodiment.

The difference from the flowchart of the second embodiment is that the supply of the first ambient air to the second end portion 14 from the fourth end portion 24 via the first filter 82 and the third end portion 22 (S214 of FIGS. 16 and S242 of FIG. 17) is replaced by the supply of the first ambient air to the second end portion 14 from the ninth end portion 92 via the first filter 82 and the third end portion 22 (S314 of FIGS. 21 and S342 of FIG. 22), and the supply of the second ambient air to the sixth end portion 34 from the eighth end portion 44 via the second filter 84 and the seventh end portion 42 (S226 of FIGS. 16 and S256 of FIG. 17) is replaced by the supply of the first ambient air to the sixth end portion 34 from the ninth end portion 92 via the first filter 82 and the seventh end portion 42 (S326 of FIGS. 21 and S356 of FIG. 22).

The atmospheric environment analysis system and the atmospheric environment analysis: method according to the present embodiment can perform environment analysis with a simple structure because one filter is used.

According to the atmospheric environment analysis system and the atmospheric environment analysis method of the present embodiment as well, an atmospheric environment analysis system and an atmospheric environment analysis method capable of efficiently performing environment analysis can be provided.

### Fourth Embodiment

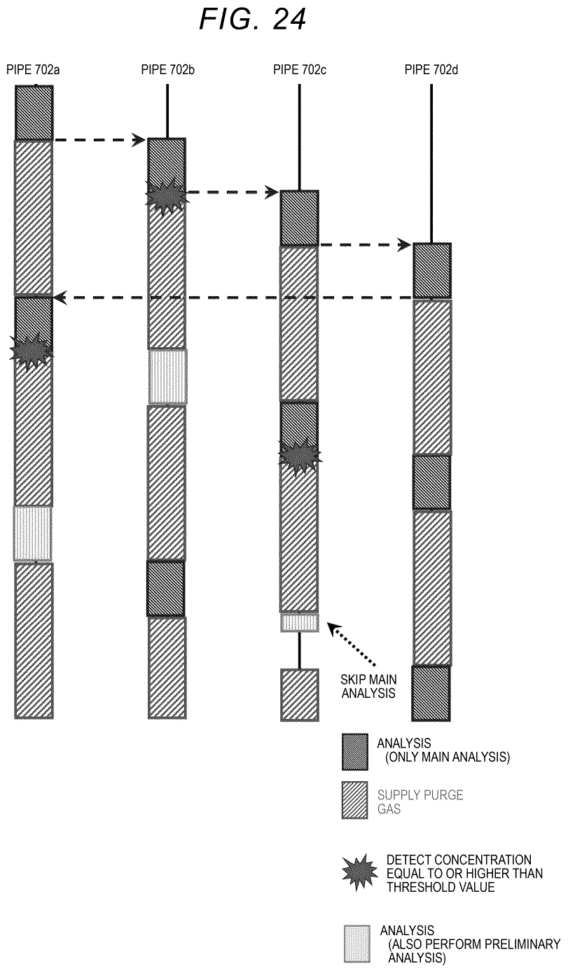

An atmospheric environment analysis system of the present embodiment has the same configuration as the atmospheric environment analysis system of the first embodiment (FIG. 1), but the method of executing the analysis of the pipe is different. More specifically, the atmospheric environment analysis system of the present embodiment includes a preliminary analysis for evaluating a state of a pipe as a result of the purge gas supply (cleaning) and a main analysis for evaluating the ambient air after the influence of the purge gas supply (cleaning) disappears as the analysis of the pipe. In this case, as shown in FIG. 23, the analysis period of the pipe includes a preliminary analysis period in which the preliminary analysis is executed, a main analysis period in which the main analysis is executed, and a switching period set between the preliminary analysis period and the main analysis period.

In the atmospheric environment analysis system of the present embodiment, the preliminary analysis is executed immediately after the purge gas is supplied and before the main analysis. In other words, the preliminary analysis is executed in an initial stage of the analysis of the pipe as a part of the analysis of the pipe.

In the atmospheric environment analysis system of the present embodiment, opening and closing states of each of the valves when the preliminary analysis is executed are the same as the opening and closing states of each of the valves when the main analysis is executed. Specifically, the opening and closing states of each of the valves when the preliminary analysis is executed for the first pipe 10 disposed at the first measurement location A1 are the same as the states shown in FIG. 11. In addition, the opening and closing states of each of the valves when the preliminary analysis is executed for the third pipe 30 disposed at the second measurement location A2 are the same as the states shown in FIG. 10.

The preliminary analysis may be executed, for example, on a certain pipe, when the concentration of the analysis target is equal to or higher than a threshold value in the analysis of the immediately preceding pipe. In other words, when the concentration of the analysis target is lower than the threshold value in the analysis of the immediately preceding pipe, only the main analysis may be executed as the analysis of the pipe, and when the concentration of the analysis target is equal to or higher than the threshold value in the analysis of the immediately preceding pipe, the preliminary analysis and the main analysis may be executed as the analysis of the pipe.

In the atmospheric environment analysis system of the present embodiment, the preliminary analysis is executed to evaluate the state of the pipe as a result of the purge gas supply (cleaning). That is, the analysis is executed to analyze the concentration of the analysis target in the purge gas when the pipe is filled with the purge gas as a result of the purge gas supply. As a result, the effect of cleaning the pipe by supplying the purge gas can be evaluated. In particular, by executing the preliminary analysis when the concentration of the analysis target in the analysis of the immediately preceding pipe is equal to or higher than the threshold value, it is possible to more reliably check that the analysis target does not remain in the pipe before the main analysis. Thereby, the execution of the main analysis in an inappropriate state may be avoided. In addition, by performing the main analysis in a state where the analysis target remains in the pipe, the analysis device 2 may be prevented from being contaminated. On the other hand, when the concentration of the analysis target in the analysis of the immediately preceding pipe is lower than the threshold value, the preliminary analysis is not executed, so that the time for the main analysis may be reserved.

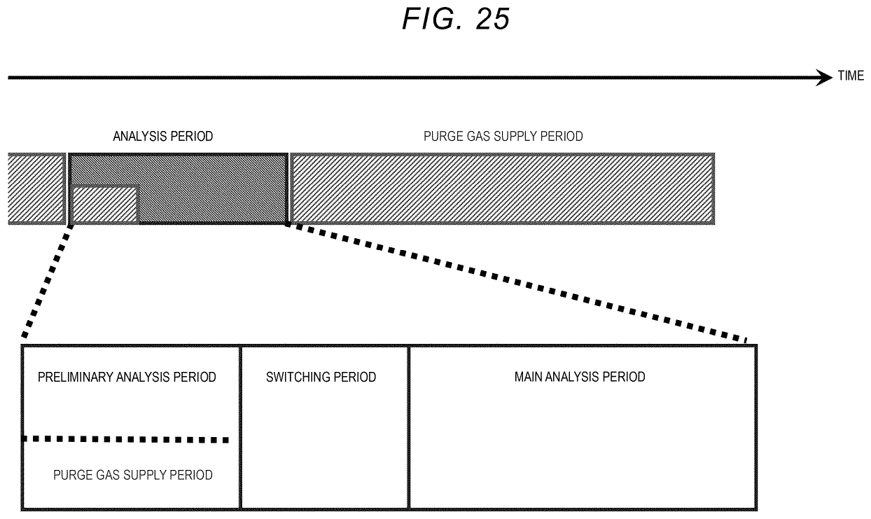

FIG. 24 is a diagram schematically showing an elapse of time of analysis of a pipe and purge gas supply (cleaning) in the atmospheric environment analysis system of the present embodiment. FIG. 24 shows a mode in which the preliminary analysis is executed only when the concentration of the analysis target is equal to or higher than the threshold value in the analysis of the immediately preceding pipe.

The preliminary analysis period may be adjusted according to, for example, the length of the pipe. Specifically, for example, in the analysis of the pipe for a long pipe, the preliminary analysis period may be lengthened, and in the analysis of the pipe for a short pipe, the preliminary analysis period may be shortened.

### Fifth Embodiment

An atmospheric environment analysis system of the present embodiment has the same configuration as the atmospheric environment analysis system of the second embodiment (FIG. 15), but the method of executing the analysis of the pipe is different. More specifically, the atmospheric environment analysis system of the present embodiment includes preliminary analysis for evaluating a state of a pipe based on a result of a pipe analysis in which purge gas is supplied (cleaning) as in the atmospheric environment analysis system of the fourth embodiment, and main analysis for evaluating an ambient air after an influence of the purge gas supply (cleaning) disappears.

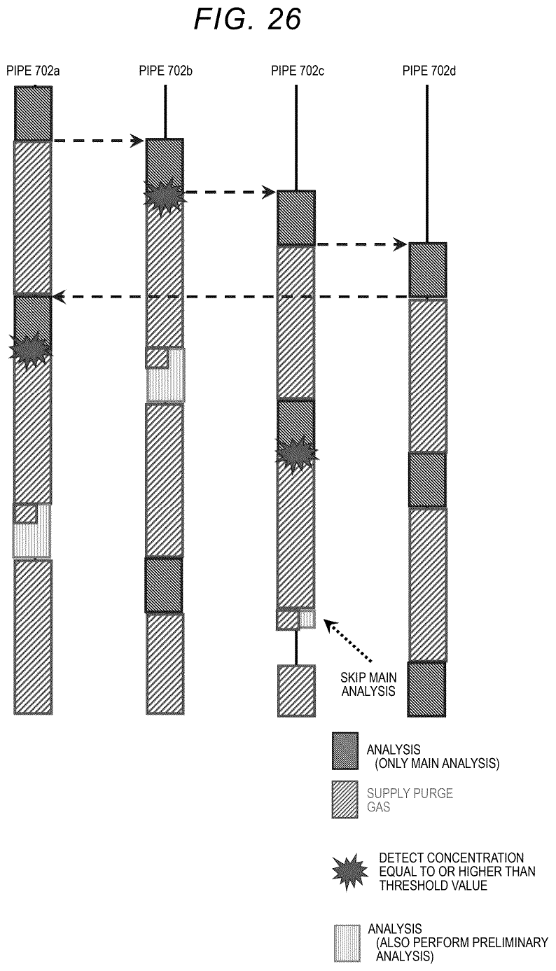

In the atmospheric environment analysis system of the fourth embodiment, the preliminary analysis was performed after the end of the purge gas supply (cleaning). In the atmospheric environment analysis system of the present embodiment, the preliminary analysis is performed in parallel with the purge gas supply (cleaning) while the purge gas supply (cleaning) is being executed, without operating the opening and closing state of the valve. In this case, as shown in FIG. 25, the analysis period of the pipe includes a preliminary analysis period in which the purge gas supply (cleaning) and the preliminary analysis are executed in parallel, a main analysis period in which the main analysis is executed, and a switching period set between the preliminary analysis period and the main analysis period.

In the atmospheric environment analysis system of the present embodiment, opening and closing states of each of the valves when the preliminary analysis is executed are the same as the opening and closing states of each of the valves when the purge gas supply (cleaning) is executed. Specifically, the opening and closing states of each of the valves when the preliminary analysis is executed for the first pipe 10 disposed at the first measurement location A1 are the same as the states shown in FIG. 18. In addition, the opening and closing states of each of the valves when the preliminary analysis is executed for the third pipe 30 disposed at the second measurement location A2 are the same as the states shown in FIG. 19.

In the atmospheric environment analysis system of the present embodiment, the effect of cleaning the pipe by the purge gas supply can be evaluated as in the atmospheric environment analysis system of the fourth embodiment.

The atmospheric environment analysis system of the present embodiment was described as having the same configuration as the atmospheric environment analysis system of the second embodiment (FIG. 15), but the atmospheric environment analysis system of the present embodiment may have the same configuration as the atmospheric environment analysis system of the third embodiment (FIG. 20). Even in this case, by setting the opening and closing states of each of the valves in the same manner, the preliminary analysis can be executed, and the effect of cleaning the pipe by supplying the purge gas can be evaluated.

FIG. 26 is a diagram schematically showing an elapse of time of analysis of a pipe and purge gas supply (cleaning) in the atmospheric environment analysis system of the present embodiment. FIG. 26 shows a manner in which the preliminary analysis is executed only when the concentration of the analysis target is equal to or higher than the threshold value in the analysis of the immediately preceding pipe.

While certain embodiments have been described, these embodiments have been presented by way of example only, and are not intended to limit the scope of the disclosure. Indeed, the novel embodiments described herein may be embodied in a variety of other forms; furthermore, various omissions, substitutions and changes in the form of the embodiments described herein may be made without departing from the spirit of the disclosure. The accompanying claims and their equivalents are intended to cover such forms or modifications as would fall within the scope and spirit of the disclosure.

What is claimed is:

1. An atmospheric environment analysis system comprising:
an analysis device;
a first pipe including:
a first end portion provided at a first measurement location;
a second end portion; and
a first location provided between the first end portion and the second end portion;
a first valve provided between the second end portion of the first pipe and the analysis device;
a gas supply source configured to supply purge gas;
a second pipe including:
a third end portion connected to the first location of the first pipe; and
a fourth end portion connected to the gas supply source;
a second valve provided in the second pipe;
a third pipe including:
a fifth end portion provided at a second measurement location;
a sixth end portion; and
a second location provided between the fifth end portion and the sixth end portion;
a third valve provided between the sixth end portion of the third pipe and the analysis device;
a fourth pipe including:
a seventh end portion connected to the second location of the third pipe; and
an eighth end portion connected to the gas supply source; and
a fourth valve provided in the fourth pipe.

2. The atmospheric environment analysis system according to claim 1,
wherein a length of the third pipe is different from a length of the first pipe.

3. The atmospheric environment analysis system according to claim 1,
wherein the purge gas is a high-purity nitrogen gas or a CDA gas.

4. The atmospheric environment analysis system according to claim 1, further comprising:
a control device configured to execute an atmospheric environment analysis method,
wherein the atmospheric environment analysis method includes:
supplying a first analysis target to the analysis device from the first measurement location via the first end portion;
performing first analysis for the first analysis target by using the analysis device;
after an elapse of a first predetermined time from a start of the first analysis and when a concentration of the first analysis target measured by the first analysis is equal to or higher than a first threshold concentration, closing the first valve, opening the second valve, and supplying the purge gas to the first measurement location from the gas supply source via the first end portion;
supplying a second analysis target to the analysis device from the second measurement location via the fifth end portion;
performing second analysis for the second analysis target by using the analysis device; and
performing, when the concentration of the first analysis target measured by the first analysis is not equal to or higher than the first threshold concentration, the second analysis without supplying the purge gas.

5. The atmospheric environment analysis system according to claim 4,
wherein the atmospheric environment analysis method includes a first analysis for evaluating a cleaning effect due to the purge gas supply, and a second analysis for evaluating an ambient air.

6. The atmospheric environment analysis system according to claim 5,
wherein, in the atmospheric environment analysis method,
after the elapse of the first predetermined time from the start of the first analysis and when the concentration of the first analysis target measured by the first analysis is equal to or higher than the first threshold concentration, the purge gas supply to the first measurement location and the second analysis are performed simultaneously.

7. The atmospheric environment analysis system according to claim 5,
wherein, in the atmospheric environment analysis method,
when the concentration of the first analysis target measured by the first analysis is equal to or higher than a third threshold concentration which is higher than the first threshold concentration, the first analysis is stopped.

8. The atmospheric environment analysis system according to claim 5,
wherein, when a concentration of the second analysis target measured by the second analysis is equal to or higher than the first threshold concentration, the third valve is closed, the fourth valve is opened, and the purge gas is supplied to the second measurement location from the gas supply source via the fifth end portion,
the first analysis target is supplied to the analysis device from the first measurement location via the first end portion,
when the concentration of the second analysis target measured by the second analysis is not equal to or higher than the first threshold concentration, the first analysis target is supplied to the analysis device from the first measurement location via the first end portion,
when the concentration of the first analysis target is equal to or lower than a second threshold concentration which is lower than the first threshold concentration, the first analysis is continued, and
when the concentration of the first analysis target is not equal to or lower than the second threshold concentration, the first valve is closed, the second valve is opened, and the purge gas is supplied to the first measurement location from the gas supply source via the first end portion.

9. The atmospheric environment analysis system according to claim 8,
wherein, when the concentration of the first analysis target is not equal to or lower than the second threshold concentration, after closing the first valve, opening the second valve, supplying the purge gas from the gas supply source to the first measurement location via the first end portion, and an elapse of a third predetermined time, the second analysis target is supplied to the analysis device from the second measurement location via the fifth end portion,
the second analysis for the second analysis target is performed by using the analysis device,
when the concentration of the second analysis target measured by the second analysis after the elapse of the third predetermined time is equal to or lower than the second threshold concentration, the second analysis after the elapse of the third predetermined time is continued, and
when the concentration of the second analysis target measured by the second analysis after the elapse of the third predetermined time is not equal to or lower than the second threshold concentration, the third valve is closed, the fourth valve is opened, and the purge gas is supplied to the second measurement location from the gas supply source via the fifth end portion.

10. An atmospheric environment analysis system comprising:
an analysis device;
a first pipe provided at a first measurement location, and including a first end portion which is an open end, and a second end portion connected to the analysis device;
a first valve provided in the first pipe;
a third pipe provided at a second measurement location, and including a fifth end portion which is an open end, and a sixth end portion connected to the analysis device;
a third valve provided in the third pipe;
a second pipe including a third end portion and a fourth end portion connected to the first pipe at a first location between the first valve and the second end portion;
the second pipe including a seventh end portion and an eighth end portion connected to the third pipe at a second location between the third valve and the sixth end portion;
a second valve provided in the second pipe;
a fourth valve provided in a fourth pipe;
a filter unit connected to the fourth end portion and the eighth end portion; and
a first pump connected to the second end portion and the sixth end portion.

11. The atmospheric environment analysis system according to claim 10,
wherein a length of the third pipe is different from a length of the first pipe.

12. The atmospheric environment analysis system according to claim 10,
wherein the analysis device has a second pump, and a capacity of the first pump is larger than a capacity of the second pump.

13. The atmospheric environment analysis system according to claim 10, further comprising:
a control device configured to execute an atmospheric environment analysis method,
wherein the atmospheric environment analysis method includes:
supplying a first analysis target to the analysis device from the first measurement location via the first end portion;
performing first analysis of the first analysis target by using the analysis device;
after an elapse of a first predetermined time from a start of the first analysis and when a concentration of the first analysis target measured by the first analysis is equal to or higher than a first threshold concentration, closing the first valve, opening the second valve, and supplying an ambient air to the second end portion via the filter unit and the third end portion;
supplying a second analysis target to the analysis device from the second measurement location via the fifth end portion;
performing second analysis for the second analysis target by using the analysis device; and
performing, when the concentration of the first analysis target measured by the first analysis is not equal to or higher than the first threshold concentration, the second analysis without supplying the ambient air.

14. The atmospheric environment analysis system according to claim 13,
wherein the first analysis includes:
a first period in which the first valve is closed, the second valve is opened, and the ambient air is supplied to the second end portion via the filter unit and the third end portion to evaluate air in the first pipe by using the analysis device; and
a second period in which the first valve is opened, the second valve is closed, and the first analysis target is supplied to the analysis device from the first measurement location via the first end portion.

15. The atmospheric environment analysis system according to claim 13,
wherein, in the atmospheric environment analysis method,
after the elapse of the first predetermined time from the start of the first analysis and when a concentration of the first analysis target measured by the first analysis is equal to or higher than the first threshold concentration, the supply of the ambient air to the second end portion and the second analysis are performed simultaneously.

16. The atmospheric environment analysis system according to claim 13,
wherein, in the atmospheric environment analysis method, when the concentration of the first analysis target measured by the first analysis is equal to or higher than a third threshold concentration which is higher than the first threshold concentration, the first analysis is stopped.

17. The atmospheric environment analysis system according to claim 13,
wherein the supply of the ambient air is performed by using the first pump.

18. The atmospheric environment analysis system according to claim 13,
wherein, when a concentration of the second analysis target measured by the second analysis is equal to or higher than the first threshold concentration, the third valve is closed, the fourth valve is opened, and the ambient air is supplied to the sixth end portion via the filter unit,
the first analysis target is supplied to the analysis device from the first measurement location via the first end portion,
when the concentration of the second analysis target measured by the second analysis is not equal to or higher than the first threshold concentration, the first analysis target is supplied to the analysis device from the first measurement location via the first end portion,
when the concentration of the first analysis target is equal to or lower than a second threshold concentration which is lower than the first threshold concentration, the first analysis is continued, and
when the concentration of the first analysis target is not equal to or lower than the second threshold concentration, the first valve is closed, the second valve is opened, and the ambient air is supplied to the second end portion via the filter unit.

19. The atmospheric environment analysis system according to claim 18,
wherein, when the concentration of the first analysis target is not equal to or lower than the second threshold concentration, after closing the first valve, opening the second valve, supplying the ambient air to the second end portion via the filter unit, and an elapse of the third predetermined time, the second analysis target is supplied to the analysis device from the second measurement location via the fifth end portion,
the second analysis for the second analysis target is performed by using the analysis device,
when the concentration of the second analysis target measured by the second analysis after the elapse of the third predetermined time is equal to or lower than the second threshold concentration, the second analysis after the elapse of the third predetermined time is continued,
when the concentration of the second analysis target measured by the second analysis after elapse of the third predetermined time is not equal to or lower than the second threshold concentration, the third valve is closed, the fourth valve is opened, and the ambient air is supplied to the sixth end portion via the filter unit.
